(19) United States
(12) Patent Application Publication
(10) Pub. No.: US 2006/0239987 A1
(43) Pub. Date: Oct. 26, 2006

(54) NUTRITIONAL COMPOSITION AND METHODS OF MAKING AND USING SAME

(76) Inventor: Robert Foster, Mesa, AZ (US)

Correspondence Address: SNELL & WILMER, 400 EAST VAN BUREN, ONE ARIZONA CENTER, PHOENIX, AZ, 85004-2202, US

(21) Appl. No.: 11/379,956

(22) Filed: Apr. 24, 2006

Related U.S. Application Data
(60) Provisional application No. 60/674,102, filed on Apr. 22, 2005.

(51) Int. Cl. A61K 38/43

(52) U.S. Cl. 424/94.1; 424/439

(57) ABSTRACT

A composition and method for treatment and/or prophylaxis of stressed-induced conditions and a method of forming the composition are disclosed. The composition includes hyaluronic acid and superoxide dismutase configured to mitigate denaturing of the superoxide dismutase in a digestive track of an animal. Mitigating the denaturing of the superoxide dismutase allows simultaneous administration of both the hyaluronic acid and superoxide dismutase to the circulatory and lymph systems of an animal.

### FIELD OF INVENTION

The present invention generally relates to nutritional compositions. More particularly, the invention relates to compositions for oral ingestion that include hyaluronic acid or a suitable salt thereof and superoxide dismutase.

### BACKGROUND OF THE INVENTION

Humans as well as other animals under physical, mental, and emotional stress, often suffer deleterious conditions such as impaired immune function, loss of lean body mass, impaired cardiovascular function and an impaired ability to repair and regenerate tissue. Each of these conditions is the result free radical damage caused by oxidative stress, accelerated metabolism, a buildup of metabolism byproducts, increased inflammation, and a reduced ability to heal.

Several drugs and supplements as well as stem cell therapies have been developed to address specific conditions or symptoms attributed to stress. Such treatments are relatively expensive, are often applied to local, stress-damaged areas after the onset of stress-caused damage, and often treat only some of the symptoms and conditions attributed to stress.

Hyaluronic acid (HA), also known as hayluron, is a glycosaminoglycan, a naturally occurring sugar, which is found in vertebrates. HA either alone or combination with other constituents, has been used to treat various symptoms of physical stress. For example, HA has been used to enhance tissue repair, stem cell migration and differentiation, cell membrane support, joint lubrication, immune system support and is a major constituent of the extracellular matrix where it contributes to structural stability (in skin, bone and many other tissues) and helps regulate hydration. HA is also a well known and effective free radical scavenger. Furthermore, HA is a transducer of shear stress generated by fluid flow through the arterial system into the production of nitric oxide (NO). HA treatments are often applied to localized areas of a body where stress-induced damage is present.

Superoxide dismutase (“SOD”), which also has been used to treat some conditions associated with stress, is a group of enzymes found in organisms in nature (humans and other organisms) that are responsible for regulating superoxide (O₂⁻) and its downstream metabolites. These enzymes are made up of various metal centers (Zn/Cu, Mn and Fe) surrounded by 3D protein structures. Superoxide is a major oxidative molecule in the body, a free radical byproduct of a body's normal metabolism; left unregulated, its constant imbalance would cause a high degree of oxidative damage. In times of stress and periods of aging, superoxide levels increase, resulting in irreversible damage to the body. The main source of this increase is from the body's own mitochondria, the energy production factory of every cell. As a body under stress needs more energy, for physical activity or increase immune function for example, the mitochondria need to metabolize glucose to produce more ATP. This process proceeds through something known as the Electron Transport Chain. As an individual mitochondria approaches peak production, some electrons escape into the cell, where they interact with and attach to oxygen, forming superoxide. The older and the more stressed the mitochondria become, the more likely they will suffer from this process and undergo their own degradation. Such degradation is called oxidative stress. These oxidation steps are driven by free radical chemistry initiated by the superoxide molecule, the result of which is a free radical induced cascade chain reaction that permanently alters the structure and function of key biological molecules. One major result of the increasing superoxide concentration and of the ensuing destructive chain reactions is a chronic condition where the body activates its overall inflammatory response in an attempt to balance the ever increasing level of damage. As the stress continues unabated, the level of damage becomes too great for the body's natural defenses to combat, and the inflammation increases to the point of “runaway condition.”

The body depends on endogenous antioxidant enzymes and molecules, as well as antioxidants gained through diet, to quell destructive free radicals. SOD, the first line of defense against superoxide, is a vital endogenous antioxidant enzyme known to produce a wide range of health benefits. Its chief function is to convert superoxide anion radicals into hydrogen peroxide and oxygen. SOD's actions against the production free radicals are important in prevention and treatment of disease and protect organs against harm caused by free radicals; combat free radical damage linked with cancer, asthma and arthritis; provide cardiovascular benefits by controlling cholesterol levels, regulating heart contractions and reducing damage caused by inflammation; promote neurological health; prevent eye problems; support immune function; and prevent exercise-induced lactic acid buildup by lessening oxidative stress and combat photon induced damage from UVA and UVB rays. Recently, innovative animal experiments have shown that certain SOD formulations can prevent cardiovascular damage under conditions of extreme stress. The mechanism of action was determined to be one involving nitrogen oxide (NO). Since levels of endogenous SOD decline with age and illness and the production of superoxide increases with age and stress, the body's capability to limit the production of and to quench free radicals responsible for initiating and accelerating disease is greatly diminished. Accordingly, it is desirable to provide a nutritional supplement that can augment the endogenous supply of SOD.

While distinct and separate applications of HA and SOD are known and may be used to treat some conditions resulting from physical and/or metal stress, it is believed that no oral composition for the simultaneous and effective use of HA and SOD to reduce conditions associated with physical, mental, and/or emotional stress, has heretofore been discovered. Accordingly, improved compositions including HA and SOD and methods of using and forming the compositions are desired.

### SUMMARY OF THE INVENTION

The present invention provides improved oral compositions that include hyaluronic acid and superoxide dismutase. While the way in which the invention addresses the various drawbacks of known compositions and methods of treating stress-induced conditions will be described in more detail below, in general, the present invention provides a composition that includes SOD and hyaluronic acid that can be delivered to and through a body simultaneously.

The compositions and methods of the present invention may be used in connection with regular, balanced diets—e.g., to supplement a diet during training, exercise, or treatment or prophylaxis of various conditions—and/or may be used in situations where such diets are not be readily available—e.g. during military combat, military training, first responder or similar situations.

In accordance with one exemplary embodiment of the present invention, a composition includes hyaluronic acid or a salt thereof and superoxide dismutase coated with a material or processed in a manner to mitigate or prevent denaturing or destruction of the SOD in the digestive track of an animal. The compositions may be in solid (e.g., pill or powder), liquid or liquid capsule form and may form part of a nutritional supplement such as a bar, drink, pill, or the like. In accordance with exemplary aspects of this embodiment, the SOD is coated with a polymer such as the wheat derivative gliadin. In accordance with further aspects of this embodiment, the composition includes additional constituents such as vitamins, minerals, antioxidants, and anti-inflammatories. By way of particular exemplary embodiments, compositions include niacin to support an increase circulation; arginine to support global production of NO; Vitamin C to, among other things, increase the production of collagen; protein source(s) to support new tissue growth; and/or carbohydrate sources. The compositions of the present invention may also include additional constituents such as fillers, stabilizers, and the like

In accordance with another exemplary embodiment of the invention, a method is provided for the treatment and/or prophylaxis of conditions resulting from physical, mental, and/or emotional stress. The method includes ingestion of a combination of HA and SOD. In accordance with various aspects of this embodiment, the method can be used to increase lean body mass, repair skin, treat osteoporosis, repair joints, recover from sports injuries, support immune functions, increase cardiovascular function, reduce inflammation, and treat other stress-related conditions.

In accordance with yet further embodiments of the invention, compositions including HA and SOD are formed by mixing SOD coated with a polymer such as gliadin with hyaluronic acid or a salt thereof.

In accordance with alternative embodiments of the invention, a method of forming the composition includes providing SOD coated with HA to mitigate denaturing of the SOD in the digestive track of an animal.

In accordance with yet further embodiments of the invention, SOD and HA are mixed together and the mixture is coated with a material to mitigate denaturing of the SOD in the digestive track of an animal. In accordance with one aspect of this embodiment of the invention, both the HA and the SOD are coated with a polymer.

### DETAILED DESCRIPTION

The present invention provides nutritional compositions or nutraceuticals for oral ingestion. The compositions include hyaluronic acid (HA) or a suitable salt thereof and superoxide dismutase (SOD). The invention also provides methods of using and making the compositions.

The composition and methods described herein can be used to supplement a diet of a variety of animals, and are particularly well suited as supplements for vertebrates such as humans. As described in more detail below, the compositions and methods of the present invention may be used to supplement a regular diet and/or may be used when a balanced diet is not readily available.

In accordance with various embodiments of the invention, a composition includes hyaluronic acid and SOD coated with a material or processed in a manner that prevents or mitigates denaturing of the SOD in the digestive track, such that the SOD and hyaluronic acid can be circulated together through the circulatory and/or lymph system of the animal, while not eliciting any antigenetic response. The simultaneous circulation of both HA and SOD has a synergistic effect of prophylaxis and treatment of stressed areas on a body because both components functionally support each other in the treatment and prophylaxis of conditions (e.g., impaired immune function, loss of lean body mass, impaired cardiovascular function, and impaired ability to repair and regenerate tissue) caused by mental, emotional, and/or physical stress. For example, HA facilitates the uptake of SOD and the free radical scavenging ability of HA supplements the ability of SOD to destroy superoxide. HA and SOD work together to, among other things, facilitate cell regeneration without interference, facilitate the rebuilding of tissue, reduce the concentration of unwanted byproducts of accelerated metabolism, limit and/or repair any damage caused by free oxygen radical and reactive oxygen species, help induce repair of structural damage by normal body mechanisms, increase circulation, enhance local repair mechanisms, and support the creation of new tissue. Thus, the combination of HA and SOD, when taken orally and circulated through a body, facilitates several elements of a recovery process in a human as well as other animals.

By way of one particular example, an oral composition including both HA and SOD reduces stress-induced damage on a circulatory system. The SOD protects the heart from stress-induced damage through an NO mechanism and HA in the epithelial lining of arteries acts as a transducer by taking the force created by the shear of moving liquid in the artery and converting that shear force into a production of NO. The properties of HA depend on its composition, configuration, and molecular weight. Various forms of HA are suitable for use with the present invention. For example, compositions of the present invention may include specific salts of HA to facilitate treatment of various conditions. In particular, calcium salts of HA may be used to treat conditions affecting bone and muscle; magnesium salts may be used for treatment of muscles and energy; silver and zinc salts may be used for anti-microbial functions; sodium salts and ammonium salts (e.g., amino acids such as arginine) may also be used in connection with the present invention. It is understood that the reference to HA throughout this specification refers to hyaluronic acid or a suitable salt thereof.

In accordance with one exemplary aspect of this embodiment, the SOD is coated with a polymer material such a gliadin. A process for forming such SOD is set forth in U.S. Pat. No. 6,045,809, the contents of which are hereby incorporated herein by reference. In accordance with various aspects of this embodiment, the SOD is derived from melons to which genes are introduced. The SOD is removed from the melons and processed to isolate the enzymes, which are then coated with gliadin. Coating the SOD with gliadin allows the SOD to pass through the stomach to the small intestine of an animal before being absorbed by the animal. In other words, the gliadin prevents the denaturing of the SOD in the digestive track of the animal. The coating also prevents or mitigates a rapid spike of SOD in the bloodstream of the animal.

In accordance with another embodiment of the invention, the HA and SOD are mixed together and the mixture is coated with a material such as the polymer gliadin to prevent denaturing of the SOD. In accordance with yet another embodiment of the invention, SOD is coated with HA to prevent denaturing of the SOD before it reaches the small intestine of an animal. In accordance with yet a further embodiment of the invention, the SOD/gliadin mixture described above is coated with HA to form an HA/SOD/gliadin mixture. And, in accordance with yet a further exemplary embodiment of the invention, HA and SOD are combined using a supercritical process to form an HA/SOD mixture. In this case, the HA/SOD combination maintains the SOD in tack until it reaches the small intestines.

Depending on the weight of the subject, the ailment to be treated, and the purpose for which the composition is used, a dose of the composition in accordance with the present invention generally includes about 10 to about 500 mg of HA, preferably about 50 to about 400 mg, and more preferably about 100 to about 300 mg of HA and about 10 to about 500 mg of SOD, preferably about 50 to about 400 mg, and more preferably about 100 to about 300 mg of the SOD. Specific exemplary preferred dosages are provided below in the examples.

In accordance with various embodiments of the invention, the dosage is selected such that appropriate turnover rate of the active ingredients is achieved. In this way, the active ingredients can be augmented by exogenous addition of the ingredients, without accumulation of the ingredients within the body. In some cases, it may be desirable to supplement a diet with the composition of the invention only under time of physical, emotional, or metal stress, while in other cases, it may be desirable to provide treatment for a prolonged period.

In accordance with various additional embodiments, the compositions of the invention include additional ingredients such as vitamins, minerals, antioxidants, and anti-inflammatory agents.

The following non-limiting examples illustrate exemplary compositions in accordance with various embodiments of the invention. These examples are merely illustrative, and it is not intended that the invention be limited to these examples. Compositions in accordance with the present invention may include the ingredients listed below as well as fillers, inert materials, flavoring agents, and preservatives typically found in compositions for treating similar conditions. In the cases where exemplary fillers, inert materials, flavoring agents, and/or preservatives are listed, these ingredients are merely exemplary, and it is understood that other similar ingredients may be substituted for the materials listed in the examples below.

The exemplary compositions listed below may be used for a variety of purposes. For example, the compositions can be used to increase strength when used in connection with exercise, facilitate faster recovery from training and exercise, increase lean body mass, maintain lean body mass in the absence of physical training, reduce a number of injuries resulting from training and exercise, promote increased cardiovascular function, reduce muscle and joint inflammation, repair skin, and enhance the immune function. As noted above, such compositions may also be desirable for the maintenance of good health and the treatment or prophylaxis of various conditions as described herein.

### EXAMPLE 1

### Joint Formula for Humans

Serving Size: Per 2 Capsules

| Amount | Preferred | | Unit of |
|---|---|---|---|
| Per Serving | Composition | Range | Measure |
| MSM | 200 | 0-1000; 150-250 | mg |
| Vitamin C | 120 | 0-500; 100-140 | mg |
| Glucosamine | 250 | 0-400; 200-300 | mg |
| SOD/Gliadin | 300 | 100-500; 250-350 | mg |
| Chondroitin | 250 | 0-500; 200-300 | mg |
| Hyaluronic Acid | 150 | 50-300; 100-200 | mg |
| Boswellic Acid | 100 | 0-200; 50-150 | mg |

*The composition may also include additional materials such as gelatin, rice flour, magnesium stearate, and silicon dioxide.

A joint formula including HA and SOD was administered to animals (humans). The use of the composition was found to relieve joint pain and facilitate healing.

### EXAMPLE 2

### Joint Formula for Dogs

Serving Size: Per 1 Tablet

| Amount | Preferred | | Unit of |
|---|---|---|---|
| Per Serving | Composition | Range | Measure |
| Vitamin C | 50 | 0-100; 25-75 | mg |
| Meat Protein | 500 | 100-1000; 400-600 | mg |
| Glucosamine | 250 | 0-500; 200-300 | mg |
| SOD/Gliadin | 100 | 25-200; 50-150 | mg |
| Chondroitin | 125 | 0-200; 100-150 | mg |
| Hyaluronic Acid | 50 | 10-100; 25-75 | mg |
| Boswellic Acid | 50 | 0-100; 25-75 | mg |

*The composition may also include additional materials such as dicalcium phosphate, stearic acid, croscarmellose dodium, silicon dioxide, magnesium stearate, and pharmaceutical glaze.

### EXAMPLE 3

### Capsule for the Creation of Lean Body

Mass Especially when Under Stress
Serving Size: Per 2 Capsules

| Amount | Preferred | | Unit of |
|---|---|---|---|
| Per Serving | Composition | Ranges | Measure |
| Vitamin A | 2500 | 0-4000; 2000-3000 | IU |
| (as Vitamin A Acetate) | | | |
| Vitamin C | 100 | 0-200; 50-150 | mg |
| (as Ascorbic Acid) | | | |
| Vitamin D | 200 | 0-300; 150-250 | IU |
| (as D-3 Cholecalciferol) | | | |
| Vitamin E | 15 | 0-30; 10-20 | IU |
| (as d-Alpha Tocopherol) | | | |
| Thiamin | 0.75 | 0-1; 0.50-0.80 | mg |
| (as Thiamin Mononitrate) | | | |
| Riboflavin | 0.85 | 0-1; 0.7-0.9 | mg |
| Niacin | 50 | 0-100; 25-75 | mg |
| (as Niacinamide) | | | |
| Vitamin B6 | 1 | 0-5; 0.5-2 | mg |
| (as Pyridoxine HCl) | | | |
| Folic Acid (as Folate) | 200 | 0-300; 100-250 | mcg |
| Vitamin B12 | 1 | 0-2; 0.5-1.5 | mcg |
| (as Cyanocobalamin) | | | |
| Biotin | 150 | 0-300; 100-200 | mcg |
| Pantothenic Acid | 5 | 0-10; 4-7 | mg |
| (as Calcium | | | |
| Pantothenate) | | | |
| Calcium | 50 | 0-100; 40-60 | mg |
| (as Whey, Caseinate) | | | |
| Iodine | 75 | 0-150; 50-100 | mcg |
| (from Potassium Iodide) | | | |
| Magnesium | 8 | 0-20; 5-10 | mg |
| (from Whey, Caseinate) | 0.02 | 0-0.1; 0.01-0.05 | mg |
| (as Magnesium Oxide) | 7.98 | 0-15; 5-10 | mg |
| Zinc | 3.75 | 0-10; 2-5 | mg |
| (from Amino Acid | | | |
| Chelate) | | | |
| Selenium | 25 | 0-50; 10-40 | mcg |
| (from Amino Acid | | | |
| Chelate) | | | |
| Copper | 1 | 0-5; 0.5-2 | mg |
| (from Amino Acid | | | |
| Chelate) | | | |
| Chromium | 50 | 0-100; 40-60 | mcg |
| (as Amino Acid | | | |
| Chelate) | | | |
| Sodium | 48 | 0-100; 30-60 | mg |
| Potassium | 30 | 0-60; 20-40 | mg |
| (from Potassium Iodide) | 0.01 | 0-0.1; 0.001-0.02 | mg |
| (from Potassium | 29.99 | 0-60; 20-50 | mg |
| Bicarbonate) | | | |
| SOD | 250 | 25-3000; 100-500 | IU |
| (as Superoxide | | | |
| Dismutase/Gliadin) | | | |
| Trehalose | 500 | 0-1000; 200-800 | mg |
| Hyaluronic Acid | 100 | 25-500; 50-150 | mg |
| MACA | 100 | 0-200; 50-150 | mg |
| Idebenone | 50 | 0-200; 40-150 | mg |
| (better than CoQ10) | | | |
| Arginine | 50 | 0-200; 40-150 | mg |
| Green Tea Extract | 50 | 0-200; 40-150 | mg |
| (50% Polyphenols) | | | |
| Citrus bioflavonoids | 50 | 0-200; 40-150 | mg |

The composition about was administered to humans and found to increase lean body mass, reduce joint pain, and increase sexual performance.

### EXAMPLE 4

### Skin Care Formula

Serving Size: Per 3 Capsules

| Amount | Preferred | | Unit of |
|---|---|---|---|
| Per Serving | Composition | Ranges | Measure |
| Vitamin C | 100 | 0-200; 50-150 | mg |
| Trace Minerals | | | |
| Iodine | 75 | 0-150; 50-100 | Mcg |
| (as potassium iodide) | | | |
| Magnesium | 8 | 0-15; 5-10 | Mg |
| Zinc (AAC) | 4 | 0-10; 2-5 | Mg |
| Selenium (AAC) | 25 | 0-50; 10-40 | Mcg |
| Copper (AAC) | 1 | 0-5; 0.5-2 | mg |
| Chromium (AAC) | 50 | 0-100; 40-60 | Mcg |
| Molybdenum | 75 | 0-150; 50-100 | Mcg |
| (as sodium molybdate) | | | |
| Blueberry extract | 250 | 50-500; 200-300 | Mg |
| SOD/GLIADIN | 250 | 50-500; 200-300 | Mg |
| ECGC-green tea | 100 | 50-250; 75-125 | Mg |
| Hyaluronic Acid | 100 | 50-250; 75-125 | Mg |
| Tocomin ® | 60 | 0-100; 50-70 | Mg |
| Boswellic Acid | 50 | 0-100; 40-60 | Mg |
| THC- | 50 | 0-100; 40-60 | Mg |
| tetrahydocucurminoids | | | |
| Centella Asiatica | 25 | 0-50; 20-30 | Mg |
| (from triterpene extract) | | | |
| Artichoke extract | 20 | 0-50; 20-30 | Mg |
| Silymarin (milk thistle) | 10 | 0-20; 5-15 | Mg |
| Venocin | 10 | 0-20; 5-15 | mg |
| (aescin, horse chesnut | | | |
| extract) | | | |

*The composition may also include additional materials such as dicalcium phosphate, stearic acid, croscarmellose dodium, silicon dioxide, magnesium stearate, and pharmaceutical glaze.

The composition of Example 4 was administered to after exposure to radiation and then surgery. The skin exhibited no necrosis and the skin around the surgical opening healed as skin that was not exposed to any radiation.

### EXAMPLE 5

### Sports Drink

Serving Size: Per 1 Packet (20 grams)

| Amount | Preferred | | Unit of |
|---|---|---|---|
| Per Serving | Composition | Ranges | Measure |
| Vitamin A (as Vitamin A Acetate) | 2500 | 0-4000; 2000-3000 | IU |
| Vitamin C (as Ascorbic Acid) | 100 | 0-200; 50-150 | mg |
| Vitamin D (as D-3 Cholecalciferol) | 200 | 0-300; 150-250 | IU |
| Vitamin E (as d-Alpha Tocopherol) | 15 | 0-30; 10-20 | IU |
| Thiamin (as Thiamin Mononitrate) | 0.75 | 0-1; 0.50-0.80 | mg |
| Riboflavin | 0.85 | 0-1; 0.7-0.9 | mg |
| Niacin (as Niacinamide) | 50 | 0-100; 25-75 | mg |
| Vitamin B6 (as Pyridoxine HCl) | 1 | 0-5; 0.5-2 | mg |
| Folic Acid (as Folate) | 200 | 0-300; 100-250 | mcg |
| Vitamin B12 (as Cyanocobalamin) | 1 | 0-2; 0.5-1.5 | mcg |
| Biotin | 150 | 0-300; 100-200 | 50% |
| Pantothenic Acid (as Calcium Pantothenate) | 5 | 0-10; 4-7 | mg |
| Calcium (as Whey, Caseinate) | 50 | 0-100; 40-60 | mg |
| Iodine (from Potassium Iodide) | 75 | 0-150; 50-200 | mcg |
| Magnesium | 8 | 0-20; 5-10 | mcg |
| (from Whey, Caseinate) | 0.03 | 0-0.1; 0.01-0.05 | mg |
| (as Magnesium Oxide) | 7.97 | 0-15; 5-10 | mg |
| Zinc (from Amino Acid Chelate) | 3.75 | 0-10; 2-5 | mg |
| Selenium (from Amino Acid Chelate) | 25 | 0-50; 10-40 | mcg |
| Copper (from Amino Acid Chelate) | 1 | 0-5; 0.5-2 | mg |
| Chromium (as Amino Acid Chelate) | 50 | 0-100; 40-60 | mcg |
| Sodium | 48 | 0-100; 40-60 | mg |
| Potassium | 30 | 0-100; 20-40 | mg |
| (from Potassium Iodide) | 0.02 | 0-0.1; 0.001-0.05 | mg |
| (from Potassium Bicarbonate) | 29.98 | 0-60; 30-50 | mg |
| SOD (as Superoxide Dismutase/Gliadin) | 1000 | 25-3000; 500-2500 | IU |
| Rice Protein Concentrate | 10000 | 2000-20000; 5000-15000 | mg |
| Trehalose | 500 | 0-1000; 250-750 | mg |
| MACA | 1000 | 0-2000; 500-1500 | mg |
| Flax Meal (Omega3, Protein, Carbs) | 500 | 0-1000; 250-750 | mg |
| Lecithin (from Phosphatidyl Lecithin) | 250 | 0-500; 200-300 | mg |
| Hyaluronic Acid | 200 | 0-500; 150-250 | mg |
| Arginine | 100 | 0-300; 75-125 | mg |
| Idebenone | 50 | 0-200; 25-100 | mg |
| Green Tea Extract (50% Polyphenols) | 50 | 0-200; 25-100 | mg |
| Citrus bioflavonoids | 50 | 0-200; 25-100 | mg |

*This composition may include natural sweeteners and flavorings as desired.

The exemplary drink mix above is added to a suitable amount of liquid (e.g. about eight ounces of water) to form a drink to, among other things, aid recovery and promote tissue repair after an exercise workout.

### EXAMPLE 6

### Sports Capsule

Serving Size: Per 2 Capsules

| Amount | Preferred | | | Unit of |
|---|---|---|---|---|
| Per Serving | Composition | | Ranges | Measure |
| Vitamin A (as Vitamin A Acetate) | 2500 | IU | 0-4000; 2000-3000 | IU |
| Vitamin C (as Ascorbic Acid) | 100 | mg | 0-200; 50-150 | mg |
| Vitamin D (as D-3 Cholecalciferol) | 200 | IU | 0-300; 150-250 | IU |
| Vitamin E (as d-Alpha Tocopherol) | 15 | IU | 0-30; 10-20 | IU |
| Thiamin (as Thiamin Mononitrate) | 0.75 | mg | 0-1; 0.50-0.80 | mg. |
| Riboflavin | 0.85 | mg | 0-1; 0.7-0.9 | mg |
| Niacin (as Niacinamide) | 50 | mg | 0-100; 25-75 | mg |
| Vitamin B6 (as Pyridoxine HCl) | 1 | mg | 0-5; 0.5-2 | mg |
| Folic Acid (as Folate) | 200 | mcg | 0-300; 100-250 | mcg |
| Vitamin B12 (as Cyanocobalamin) | 1 | mcg | 0-2; 0.5-1.5 | mcg |
| Biotin | 150 | mcg | 0-300; 100-200 | 50% |
| Pantothenic Acid (as Calcium | 5 | mg | 0-10; 4-7 | mg |
| Pantothenate) | | | | |
| Calcium (as Whey, Caseinate) | 50 | mg | 0-100; 40-60 | mg |
| Iodine (from Potassium Iodide) | 75 | mcg | 0-150; 50-200 | mcg |
| Magnesium | 8 | mg | 0-20; 5-10 | mg |
| (from Whey, Caseinate) | 0.02 | mg | 0-0.1; 0.01-0.05 | mg |
| (as Magnesium Oxide) | 7.98 | mg | 0-15; 5-10 | mg |
| Zinc (from Amino Acid Chelate) | 3.75 | mg | 0-10; 2-5 | mg |
| Selenium (from Amino Acid Chelate) | 25 | mcg | 0-50; 10-40 | mcg |
| Copper (from Amino Acid Chelate) | 1 | mg | 0-5; 0.5-2 | mg |
| Chromium (as Amino Acid Chelate) | 50 | mcg | 0-100; 40-60 | mcg |
| Sodium | 48 | mg | 0-100; 40-60 | mg |
| Potassium | 30 | mg | 0-50; 20-40 | mg |
| (from Potassium Iodide) | 0.01 | mg | 0-0.001; 0.001-0.02 | mg |
| (from Potassium Bicarbonate) | 29.99 | mg | 0-60; 30-50 | mg |
| SOD (as Superoxide Dismutase/Gliadin) | 250 | IU | 25-3000; 100-500 | IU |
| Trehalose | 50 | mg | 0-1000; 20-800 | mg |
| Hyaluronic Acid | 100 | mg | 25-500; 50-150 | mg |
| MACA | 100 | mg | 0-200; 50-150 | mg |
| Idebenone (better than CoQ10) | 50 | mg | 0-200; 40-150 | mg |
| Arginine | 50 | mg | 0-200; 40-150 | mg |
| Green Tea Extract (50% Polyphenols) | 50 | mg | 0-200; 40-150 | mg |
| Citrus bioflavonoids | 50 | mg | 0-200; 40-150 | mg |

### EXAMPLE 7

### Sun Damage Repair and Support

Serving Size: Per 3 Capsules

| Amount | Preferred | | Unit of |
|---|---|---|---|
| Per Serving | Composition | Ranges | Measure |
| Vitamin C | 100 | 0-200; 50-150 | mg |
| Trace Minerals | | | |
| Iodine (as potassium iodide) | 75 | 0-150; 50-100 | mcg |
| Magnesium | 8 | 0-15; 5-10 | mg |
| Zinc (AAC) | 4 | 0-10; 2-5 | mg |
| Selenium (AAC) | 25 | 0-50; 10-40 | mcg |
| Copper (AAC) | 1 | 0-5; 0.5-2 | mg |
| Chromium (AAC) | 50 | 0-100; 40-60 | mcg |
| Molybdenum (as sodium molybdate) | 75 | 0-150; 50-100 | mcg |
| Blueberry extract | 250 | 50-500; 200-300 | mg |
| SOD/GLIADIN | 250 | 50-500; 200-300 | mg |
| ECGC-green tea | 100 | 50-250; 75-125 | mg |
| Hyaluronic Acid | 100 | 50-250; 75-125 | mg |
| Tocomin ® | 60 | 0-100; 50-70 | mg |
| Boswellic Acid | 50 | 0-100; 40-60 | mg |
| THC-tetrahydocucurminoids | 50 | 0-100; 40-60 | mg |
| Centella Asiatica (from triterpene extract) | 25 | 0-50; 20-30 | mg |
| Artichoke extract | 20 | 0-50; 20-30 | mg |
| Silymarin (milk thistle) | 10 | 0-20; 5-15 | mg |
| Venocin (aescin, horse chesnut extract) | 10 | 0-20; 5-15 | mg |

*The composition may also include additional materials such as dicalcium phosphate, stearic acid, croscarmellose dodium, silicon dioxide, magnesium stearate, and pharmaceutical glaze.

### EXAMPLE 8

### Cardiovascular Support

| Amount | Preferred | | Unit of |
|---|---|---|---|
| Per Serving | Composition | Ranges | Measure |
| SOD/Gliadin | 250 | 25-500; 200-300 | mg |
| Hyaluronic Acid | 150 | 10-500; 100-250 | mg |
| Vitamin C | 500 | 50-1000; 200-800 | mg |
| Niacin | 75 | 25-200; 40-150 | mg |
| Folic Acid | 200 | 50-500; 100-300 | mcg |
| Arginine | 50 | 25-100; 30-75 | mg |
| Alpha Lipoic | 100 | 50-400; 75-300 | mg |
| Acid | | | |
| Vitamin B Complex | 100 | 10-1000; 50-400 | mg |
| MACA | 100 | 50-500; 75-150 | mg |

An SOD/HA composition was also administered as an adjunct to surgical wound healing of a torn bicep. The patient recovered quickly and was able to lift weights again within eight weeks after the surgery.

The SOD/HA compositions of the present invention have also been used to and been found efficacious for regulating blood sugar in diabetics, supporting diabetics as they lost weight, mitigating any need for pain medication, and mitigating neuropathy.

The compositions of the invention may be formed in a variety of ways. For example, the gliadin-coated SOD may be mixed with the HA (in powder form) and then the remaining ingredients may be added to the SOD/HA mixture.

Although exemplary embodiments of the present invention are set forth herein, it should be appreciated that the invention is not so limited. Various modifications, variations, and enhancements in composition and method set forth herein may be made without departing from the spirit and scope of the present invention.

I claim:

1. A oral composition for treatment or prophylaxis of a stress-induced condition of an animal, the composition comprising:
hyaluronic acid or a suitable salt thereof;
superoxide dismutase; and
a coating configured to mitigate denaturing of the superoxide dismutase in a digestive track of the animal.

2. The composition of claim 1, wherein the coating is in the form of a polymer.

3. The composition of claim 2, wherein the coating comprises gliadin.

4. The composition of claim 1, wherein the coating covers a mixture of the hyaluronic acid or suitable salt thereof and the superoxide dismutase.

5. The composition of claim 1, wherein the salt comprises a moiety selected from the group consisting of calcium, magnesium, silver, zinc, sodium, and ammonium.

6. The composition of claim 1, wherein the coating comprises hyaluronic acid.

7. The composition of claim 1, further comprising a constituent selected from the group consisting of vitamins, minerals, antioxidants, and anti-inflammatory agents.

8. The composition of claim 1, wherein the hyaluronic acid is present in an amount of about 10 mg to about 500 mg.

9. The composition of claim 1, wherein the superoxide dismutase is present in an amount of about 10 mg to about 500 mg.

10. A method of supplementing a diet for treatment or prophylaxis of a stress-induced condition, the method comprising the steps of:
providing a composition comprising hyaluronic acid and superoxide dismutase; and
orally ingesting the composition.

11. The method of claim 10, wherein the step of providing comprises providing superoxide dismutase coated with a polymer.

12. The method of claim 11, wherein the polymer comprises gliadin.

13. The method of claim 11, wherein the polymer comprises hyaluronic acid.

14. The method of claim 10, wherein the step of orally ingesting comprises ingesting a fluid.

15. The method of claim 10, wherein the step of orally ingesting comprises ingesting a solid.

16. The method of claim 10, wherein the step of providing further comprises providing a constituent selected from the group consisting of vitamins, minerals, antioxidants, and anti-inflammatories.

17. The method of claim 10, wherein the treatment comprises a treatment selected from the group consisting of repair skin damage, increase circulation, increase the production of collagen, support new tissue growth, increase lean body mass, treat osteoporosis, reduce inflammation, support immune functions, repair joints, and recover from injuries.

18. A method of forming a composition for oral ingestion, the method comprising the steps of:
providing superoxide dismutase;
coating the superoxide dismutase with a material to mitigate denaturing of the superoxide dismutase in a digestive track of an animal; and
mixing the superoxide dismutase with hyaluronic acid.

19. The method of claim 18, wherein the step of coating comprises spraying the superoxide dismutase with a polymer.

20. The method of claim 19, wherein the polymer comprises gliadin.
